(19) United States
(12) Patent Application Publication
(10) Pub. No.: US 2023/0073663 A1
(43) Pub. Date: Mar. 9, 2023

(54) DEVICE AND METHOD FOR RECEIVING BIOSIGNAL

(71) Applicant: ELECTRONICS AND TELECOMMUNICATIONS RESEARCH INSTITUTE, Daejeon (KR)

(72) Inventors: Kwang IL OH, Daejeon (KR); Hyung-IL PARK, Daejeon (KR); Hyuk KIM, Daejeon (KR)

(21) Appl. No.: 17/845,681

(22) Filed: Jun. 21, 2022

(30) Foreign Application Priority Data
Sep. 3, 2021 (KR) 10-2021-0117338

(51) Int. Cl. H04B 13/00; H04L 7/00

(52) U.S. Cl. CPC H04B 13/005; H04L 7/0016

(57) ABSTRACT

Disclosed are a biosignal receiving device and a method thereof. The biosignal receiving device used in a communication system using a human body as a medium includes a receiving electrode unit including a plurality of receiving electrodes, an input selection unit including two MUXs for selecting one of the plurality of receiving electrodes, and that output biosignals received by the selected electrodes, a filter that removes noise included in the biosignals and output filtered signals from which the noise is removed, a differential amplifier that amplifies a difference between the filtered signals and output amplified signals, a CDR circuit that generates a data signal and a clock signal from the amplified signals and outputs the data signal and the clock signal, and a controller that output selection signals for selecting one of the plurality of receiving electrodes, based on the data signal and the clock signal.

### CROSS-REFERENCE TO RELATED APPLICATIONS

This application claims priority under 35 U.S.C. § 119 to Korean Patent Application No. 10-2021-0117338, filed on Sep. 3, 2021, in the Korean Intellectual Property Office, the disclosures of which are incorporated by reference herein in their entireties.

### BACKGROUND

Embodiments of the present disclosure described herein relate to communication technology, and more particularly, relate to a biosignal receiving device and method using an optimal electrode combination selected from among a plurality of receiving electrodes.

Human body communication using a human body as a medium is known as one of communication methods for transmitting signals and information from a transmitting device to a receiving device. In human body communication, since signals are transmitted using the human body as a medium, binding force on signals can be exerted. In addition, as transmission and receiving operations can be performed by directly transmitting a digital signal to the human body without requiring signal modulation and demodulation, the human body communication can be implemented as a communication system having a simple structure but low power consumption.

In a communication system using the human body as a medium, the principle of transmitting a signal from the transmitting device to the human body may be based on an electric field formed between the earth ground and a ground included in the transmitting device and an electric field formed between the human body and a transmitting electrode. In detail, when a time-varying voltage is applied to the transmitting electrode, a time-varying electric field corresponding thereto may be formed from the transmitting electrode to the human body, and time-varying charges may be also generated in the human body, such that a signal may be transmitted from the transmitting device to the human body. In addition, in a communication system using the human body as a medium, the principle of transmitting a signal from the human body to the receiving device is similar to the above description, and ultimately, communication between the transmitting device and the receiving device can be performed through this series of mechanisms.

### SUMMARY

Embodiments of the present disclosure provide a biosignal receiving device and method capable of using an optimal electrode combination selected from among a plurality of receiving electrodes.

According to an embodiment of the present disclosure, a biosignal receiving device used in a communication system using a human body as a medium, which includes a receiving electrode unit including a plurality of receiving electrodes for receiving a biosignal received from the human body, an input selection unit including a first MUX for selecting a first electrode from among the plurality of receiving electrodes and a second MUX for selecting a second electrode different from the first electrode from among the plurality of receiving electrodes, and that outputs a first biosignal received by the first electrode and a second biosignal received by the second electrode, a filter that removes noise included in the first biosignal and the second biosignal and outputs a first filtered signal and a second filtered signal from which the noise is removed, a differential amplifier that amplifies a difference between the first filtered signal and the second filtered signal and outputs a first amplified signal and a second amplified signal, a CDR circuit that generates a data signal and a clock signal from the first amplified signal and the second amplified signal and outputs the data signal and the clock signal, and a controller that outputs a first selection signal for selecting the first electrode from among the plurality of receiving electrodes and a second selection signal for selecting the second electrode from among the plurality of receiving electrodes to the input selection unit, based on the data signal and the clock signal.

According to an embodiment, the controller may include a correlation value detector that detects a correlation value of the biosignal based on the data signal and the clock signal and outputs the correlation value, and an electrode pair selector that generates the first selection signal and the second selection signal based on the clock signal and the correlation value.

According to an embodiment, the correlation value detector may compare a preamble of a transmitting signal transmitted from a transmitting device included in a communication system using the human body as the medium with a preamble of the biosignal to derive the correlation value.

According to an embodiment, when specific electrodes among the plurality of electrodes are selected as the first electrode and the second electrode, and the correlation value exceeds a predetermined threshold correlation value, the specific electrodes may be finally selected as the first electrode and the second electrode.

According to an embodiment, the biosignal receiving device may further include a reference voltage level detector that determines whether a level of the biosignal is equal to or greater than a level of a reference voltage based on the first amplified signal, and outputs a pulse signal to the controller based on the determination result.

According to an embodiment, the controller may generate, based on the pulse signal, a result signal indicating a result of determining whether the biosignal has the level greater than or equal to the reference voltage, and may output the result signal to the electrode pair selector.

According to an embodiment, the electrode pair selector may generate the first selection signal and the second selection signal based on the correlation value and the result of the determining.

According to an embodiment, a reference voltage level detector may include an amplifier that amplifies and outputs a voltage applied to a node between a supply voltage and a ground, and a comparator that compares the first amplified signal with a reference voltage output from the amplifier and outputs the pulse signal corresponding to the comparison result.

According to an embodiment, the pulse signal may have a high level when the level of the first amplified signal is greater than or equal to a level of the reference voltage, and may have a low level when the level of the first amplified signal is less than the reference voltage.

According to an embodiment of the present disclosure, a biosignal receiving method includes selecting a first electrode and a second electrode from among a plurality of receiving electrodes, receiving a biosignal from the first electrode and the second electrode, detecting a correlation value with respect to the biosignal, and determining whether a level of the biosignal is greater than or equal to a level of a reference voltage, and the correlation value is based on a correlation between a preamble of a transmitting signal transmitted to a human body providing the biosignal and a preamble of the biosignal.

According to an embodiment, the method further includes determining whether the correlation value is detected for all of receiving electrode combinations generated from the plurality of receiving electrodes.

According to an embodiment, the method further includes, when the correlation value is not detected for all of the receiving electrode combinations, reselecting the combination of the first electrode and the second electrode from receiving electrode combinations that are not selected among the receiving electrode combinations.

According to an embodiment, the method further includes determining whether the correlation value is greater than or equal to a predetermined threshold correlation value.

### BRIEF DESCRIPTION OF THE FIGURES

The above and other objects and features of the present disclosure will become apparent by describing in detail embodiments thereof with reference to the accompanying drawings.

FIG. 1 is a block diagram illustrating a biosignal receiving device according to an embodiment of the present disclosure.

FIG. 2 is a block diagram illustrating a controller included in a biosignal receiving device illustrated in FIG. 1.

FIG. 3A is a diagram illustrating an embodiment in which four receiving electrodes are included in a biosignal receiving device illustrated in FIG. 1.

FIG. 3B is a diagram for describing a principle of selecting a receiving electrode combination in a biosignal receiving device illustrated in FIG. 3A.

FIG. 4 is a block diagram illustrating a biosignal receiving device according to another embodiment of the present disclosure.

FIG. 5 is a block diagram illustrating a controller included in a biosignal receiving device illustrated in FIG. 4.

FIG. 6 is a block diagram illustrating a reference voltage level detector included in the biosignal receiving device illustrated in FIG. 4.

FIG. 7A is a diagram illustrating an embodiment in which four receiving electrodes are included in a biosignal receiving device illustrated in FIG. 2.

FIG. 7B is a diagram for describing a principle of selecting a receiving electrode combination in a biosignal receiving device illustrated in FIG. 7A.

FIG. 8 is a diagram illustrating a method of receiving a biosignal according to an embodiment of the present disclosure.

### DETAILED DESCRIPTION

Hereinafter, embodiments of the present disclosure will be described clearly and in detail such that those skilled in the art may easily carry out the present disclosure.

The terms used in the specification are provided to describe the embodiments, not to limit the inventive concept. As used in the specification, the singular terms “a”, “an” and “the” are intended to include the plural forms as well, unless the context clearly indicates otherwise. The terms “comprises” and/or “comprising” when used in the specification, specify the presence of steps, operations, elements, and/or components, but do not preclude the presence or addition of one or more other steps, operations, elements, components, and/or groups thereof

In the specification, the term “first and/or second” will be used to describe various elements but will be described only for the purpose of distinguishing one element from another element, not limiting an element of the corresponding term. For example, without departing the scope of the inventive concept, a first element may be referred to as a second element, and similarly, a second element may be referred to as a first element.

Unless otherwise defined, all terms (including technical and scientific terms) used in the specification should have the same meaning as commonly understood by those skilled in the art to which the inventive concept pertains. The terms, such as those defined in commonly used dictionaries, should not be interpreted in an idealized or overly formal sense unless expressly so defined herein. The same reference numerals represent the same elements throughout the specification.

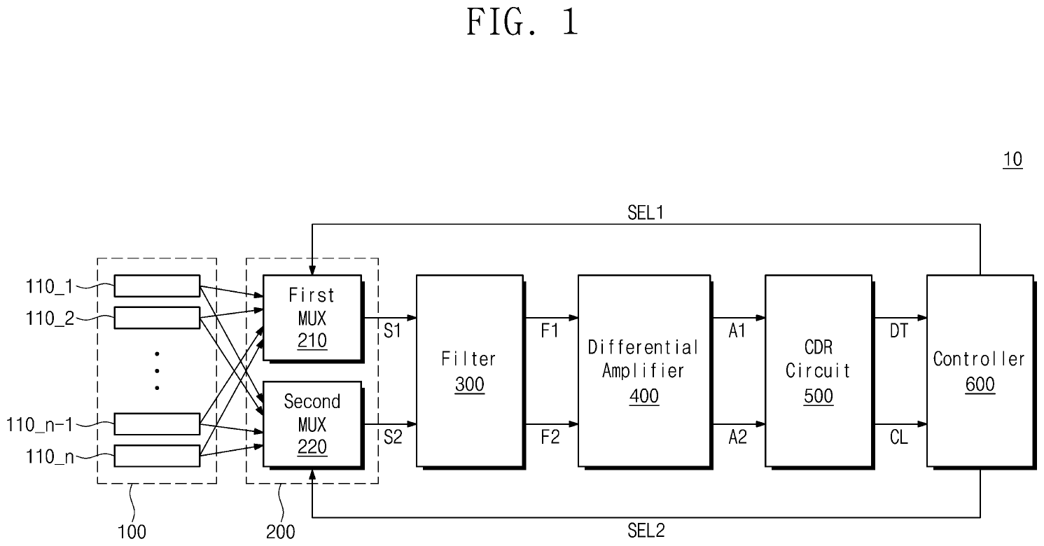

FIG. 1 is a block diagram illustrating a biosignal receiving device 10 according to an embodiment of the present disclosure. The biosignal receiving device 10 illustrated in FIG. 1 may be included in a human body communication system and may communicate with a transmitting device included in the human body communication system. In the human body communication system, the transmitting device may transmit a signal to a human body, and the transmitted signal may be provided to the biosignal receiving device 10 through the human body. For example, a transmitting electrode of the transmitting device may be attached to the epidermis of a human body to transmit a signal, and the form of the transmitting signal may be a baseband signal of a square wave. A signal that is transmitted to the biosignal receiving device 10 through a human body medium is basically in the form of a baseband signal of a square wave. However, since signal attenuation due to the human body medium and distortion due to noises around the human body may occur, the signal may be provided to the biosignal receiving device 10 in the form of a distorted signal.

Referring to FIG. 1, the biosignal receiving device 10 may include a receiving electrode unit 100, an input selection unit 200, a filter 300, a differential amplifier 400, a clock and data recovery (hereinafter referred to as a CDR) circuit 500, and a controller 600. The receiving electrode unit 100 may include a first receiving electrode 110_1 to an n-th receiving electrode 110_n, and the input selection unit 200 may include a first MUX 210 and a second MUX 220.

Each of the first to n-th receiving electrodes 110_1, 110_2, . . . , and 110_n included in the receiving electrode unit 100 may receive a signal provided from the transmitting device through the human body. Each of the first to n-th receiving electrodes 110_1, 110_2, . . . , and 110_n may provide the received signal to the first MUX 210 and the second MUX 220 included in the input selection unit 200.

The input selection unit 200 may select some signals from among a plurality of signals provided from the receiving electrode unit 100. The first MUX 210 and the second MUX 220 included in the input selection unit 200 may independently perform a selection operation. However, the first MUX 210 and the second MUX 220 included in the input selection unit 200 cannot select a signal received from the same receiving electrode. For example, when the first MUX 210 selects a receiving signal received from the first receiving electrode 110_1, the second MUX 220 cannot select the receiving signal received from the first receiving electrode 110_1.

A receiving electrode selected by the first MUX 210 from among the first to n-th receiving electrodes 110_1, 110_2, . . . , and 110_n may correspond to a first electrode of the biosignal receiving device 10, and a receiving electrode selected by the second MUX 220 from among the first to n-th receiving electrodes 110_1, 110_2, . . . , 110_n may correspond to a second electrode of the biosignal receiving device 10. In the biosignal receiving device 10, the first electrode may be in contact with human skin. The second electrode may be used as a ground of the biosignal receiving device 10. A first receiving signal Si of the first electrode selected by the first MUX 210 and a second receiving signal S2 of the second electrode selected by the second MUX 220 may be provided to the filter 300.

The filter 300 may perform a filtering operation on the first receiving signal S1 and the second receiving signal S2. For example, the filter 300 may pass or block only signals within a specific bandwidth from the first receiving signal Si and the second receiving signal S2. The filter 300 may cancel noise through the filtering operation on the first receiving signal Si and the second receiving signal S2. A first filtered signal F1 obtained by filtering the first receiving signal Si and a second filtered signal F2 obtained by filtering the second receiving signal S2 may be provided to the differential amplifier 400.

The differential amplifier 400 may amplify and output a difference between a positive input and a negative input of the differential amplifier 400. In other words, the differential amplifier 400 may amplify the difference between the first filtered signal F1 and the second filtered signal F2 and may output the amplified signal. The first filtered signal F1 may be provided to the positive input of the differential amplifier 400, and the second filter circuit F2 may be provided to the negative input of the differential amplifier 400.

The first filtered signal F1 may correspond to a voltage induced in the first electrode, and the voltage induced in the first electrode may be generated by an electric field formed between the human body and the receiving electrode. Also, the second filtered signal F2 may correspond to a voltage induced in the second electrode, and the voltage induced in the second electrode may be generated by an electric field formed between the second electrode and the ground of a ground surface. The voltage induced in the second electrode may be used as a reference voltage for amplifying the voltage induced in the receiving electrode. The differential amplifier 400 may provide a first amplified signal Al and a second amplified signal A2 to the CDR circuit 500.

The CDR circuit 500 may separate a data signal DT and a clock signal CL from the first amplified signal Al and the second amplified signal A2. The CDR circuit 500 may provide the data signal DT and the clock signal CL to the controller 600.

The controller 600 may determine optimal receiving electrode combinations for human body communication with respect to the first to n-th receiving electrodes 110_1, 110_2, . . . , 110_n included in the receiving electrode unit 100, based on the data signal DT and the clock signal CL. The controller 600 may allow the input selection unit 200 to select optimal receiving electrode combinations, by providing a first selection signal SEL1 and a second selection signal SEL2 based on the determination result of the optimal receiving electrode combinations to the input selection unit 200.

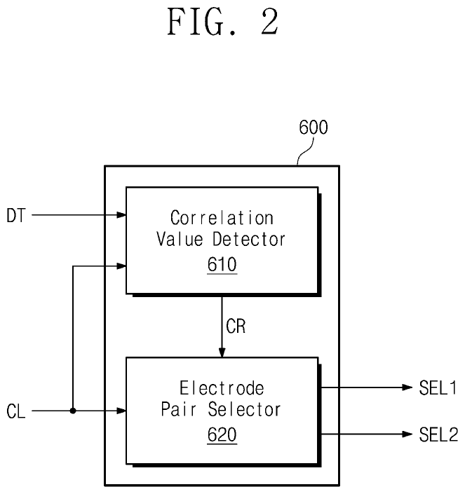

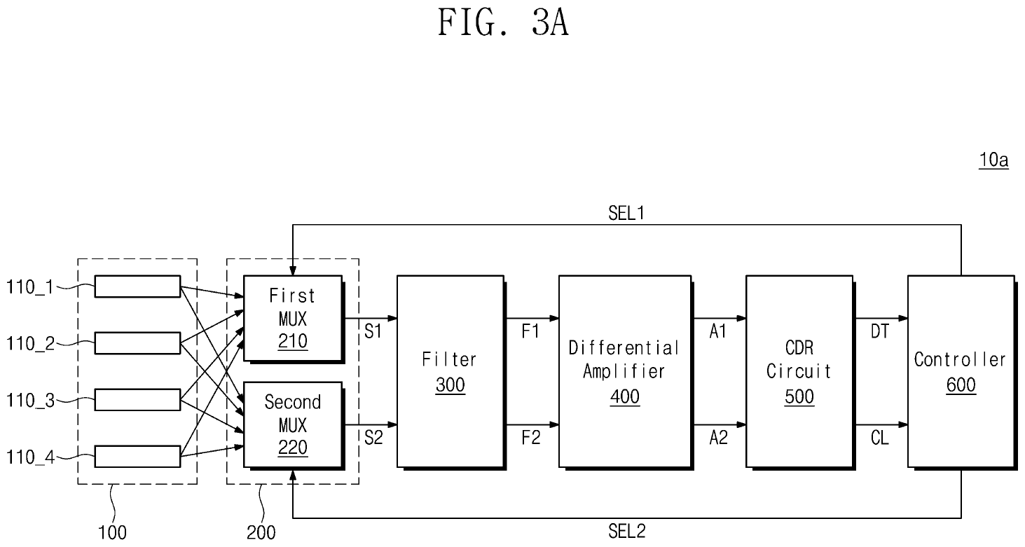

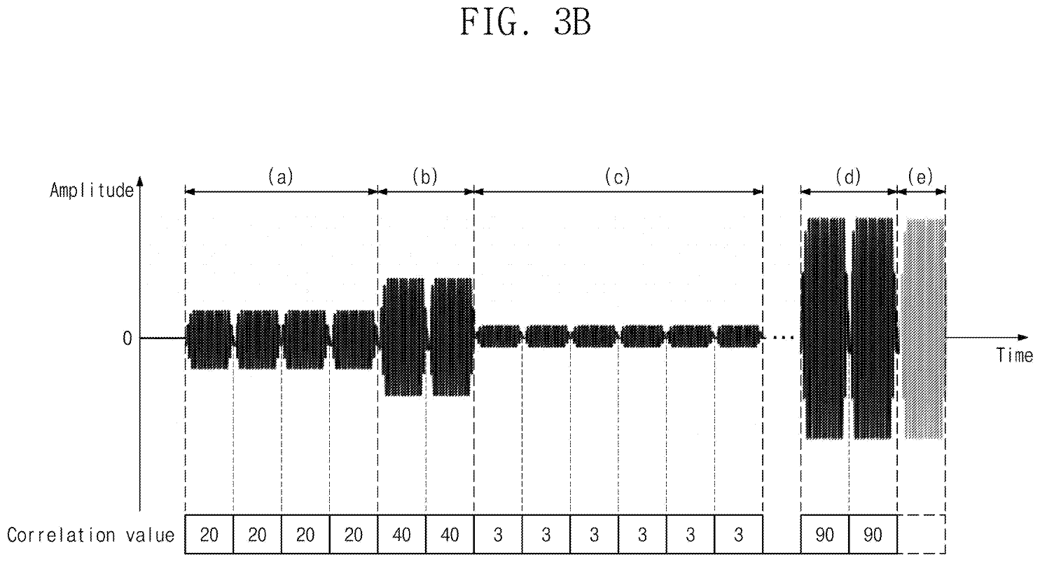

In an embodiment according to the present disclosure, the biosignal receiving device 10 may allow to selectively operate a receiving electrode capable of providing an optimal signal quality among the plurality of receiving electrodes 110_1, 110_2, . . . , 110_n, thereby improving the performance of the human body communication system. A method of selecting the optimal receiving electrode combinations in the biosignal receiving device 10 according to the present disclosure and the configuration of the controller 600 for this will be described in detail with reference to FIGS. 2 to 3B to be described later.

FIG. 2 is a block diagram illustrating the controller 600 included in the biosignal receiving device illustrated in FIG. 1. Referring to FIG. 2, the controller 600 may include a correlation value detector 610 and an electrode pair selector 620.

The correlation value detector 610 of the controller 600 may receive the data signal DT and the clock signal CL from the CDR circuit 500 (refer to FIG. 1). The correlation value detector 610 may detect a correlation value based on the data signal DT and the clock signal CL. The correlation value means a value corresponding to a preamble match degree of a signal received to the biosignal receiving device 10 (refer to FIG. 1) with respect to the preamble signal transmitted from the transmitting device in the human body communication system.

For example, when the signal transmitted from the transmitting device is transmitted to the biosignal receiving device 10 without attenuation or error, the preamble of the transmitting signal and the preamble of the receiving signal may have the same data stream, and in this case, the correlation value may be a value of 100. In contrast, when the signal transmitted from the transmitting device is transmitted to the biosignal receiving device 10 with attenuation or error, the preamble of the transmitting signal and the preamble of the receiving signal may have different data streams, and in this case, the correlation value may have a value greater than or equal to 0 and less than 100. The correlation value detected by the correlation value detector 610 may be provided to the electrode pair selector 620.

The electrode pair selector 620 may select an optimal electrode pair based on the clock signal CL provided from the CDR circuit 500 and the correlation value provided from the correlation value detector 610. The optimal electrode pair means a receiving electrode combination capable of exhibiting optimal communication performance among the first to n-th receiving electrodes 110_1, 110_2, . . . , 110_n included in the receiving electrode unit 100 (refer to FIG. 1). The electrode pair selector 620 may output the first selection signal SEL1 including information on the receiving electrode to operate as the first electrode and the second selection signal SEL2 including information on the receiving electrode to operate as the second electrode to the input selection unit 200 (refer to FIG. 1). A method of selecting an optimal receiving electrode combination will be described in detail with reference to FIGS. 3A and 3B, which will be described later.

FIG. 3A is a diagram illustrating an embodiment 10a in which the four receiving electrodes 110_1, 110_2, 110_3, and 110_4 are included in the biosignal receiving device 10 (refer to FIG. 1) illustrated in FIG. 1. FIG. 3B is a diagram for describing a principle of selecting a receiving electrode combination in a biosignal receiving device illustrated in FIG. 3A. Hereinafter, descriptions of components and operations of the biosignal receiving device 10 according to the embodiment of the present disclosure described above with reference to FIGS. 1 to 2 will be omitted to avoid redundancy.

There are 12 receiving electrode pairs (the first electrode and the second electrode) that may occur in the embodiment 10a in which the four receiving electrodes 110_1, 110_2, 110_3, and 110_4 illustrated in FIG. 3A are included. Accordingly, the biosignal receiving device 10 according to an embodiment of the present disclosure may select a receiving electrode pair capable of exhibiting optimal communication performance among 12 cases.

FIG. 3B illustrates a waveform received by the biosignal receiving device 10 according to an embodiment of the present disclosure. In more detail, FIG. 3B illustrates a waveform of the receiving signal received from some of the receiving electrode pairs among the receiving electrode pairs (the first electrode and the second electrode) that may occur in the embodiment 10a in which the four receiving electrodes 110_1, 110_2, 110_3, and 110_4 illustrated in FIG. 3A are included. In a graph illustrating the waveform of the receiving signal, an x-axis means a time, and a y-axis means an amplitude of the receiving signal.

For example, in FIG. 3B, the signal in section (a) may illustrate a waveform of the signal received from the first receiving electrode 110_1 and the second receiving electrode 110_2, and the signal in section (b) may illustrate a waveform of the signal received from the first receiving electrode 110_1 and the fourth receiving electrode 110_4. The signal in section (c) may illustrate a waveform of the signal received from the second receiving electrode 110_2 and the first receiving electrode 110_1, and the signal in section (d) may illustrate a waveform of the signal received from the third receiving electrode 110_3 and the fourth receiving electrode 110_4. The signal in section (e) may be a data signal.

In the embodiment 10a in which the four receiving electrodes 110_1, 110_2, 110_3, and 110_4 illustrated in FIG. 3A are included, the correlation value detector 610 included in the controller 600 (refer to FIG. 1) may derive correlation values for each case. In FIG. 3B, it may be seen that the correlation value in section (a) is derived as 20, which means that a match rate between the preamble of the transmitting signal transmitted from the transmitting device and the preamble of the receiving signal received by the biosignal receiving device 10 is 20% when the first receiving electrode 110_1 is selected as the first electrode and the second receiving electrode 110_2 is selected as the second electrode. Accordingly, as in section (b), when the first receiving electrode 110_1 is selected as the first electrode and the fourth receiving electrode 110_4 is selected as the second electrode, it may be confirmed that the correlation value is 40. In the case of section (c), it may be seen that the correlation value is 3, and in the case of section (d), it may be seen that the correlation value is 90.

Based on these results, the electrode pair selector 620 may select an optimal electrode pair based on the correlation value. As an example, the electrode pair selector 620 may derive a correlation value associated with the number of all 12 cases that may occur in the embodiment 10a in which the four receiving electrodes 110_1, 110_2, 110_3, and 110_4 illustrated in FIG. 3A are included, and may select a receiving electrode combination having a maximum correlation value. Alternatively, the electrode pair selector 620 may select a receiving electrode combination having a correlation value exceeding a threshold correlation value based on the predetermined threshold correlation value.

Also, in an embodiment according to the present disclosure, the biosignal receiving device 10 may recognize data received after the preamble of the receiving signal received by the receiving electrode combination having the maximum correlation value as data transmitted by the transmitting device.

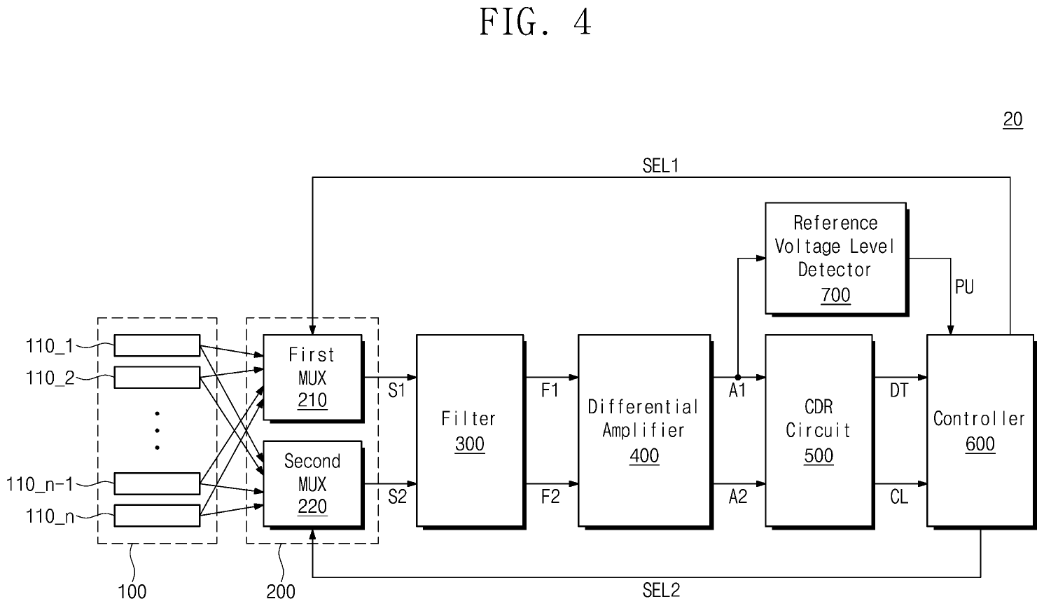

FIG. 4 is a block diagram illustrating a biosignal receiving device 20, according to another embodiment of the present disclosure. Hereinafter, descriptions of components and operations of the biosignal receiving device 10 according to the embodiment of the present disclosure described above with reference to FIGS. 1 to 3B will be omitted to avoid redundancy. Referring to FIG. 4, the biosignal receiving device 20 according to another embodiment of the present disclosure may further include a reference voltage level detector 700.

The reference voltage level detector 700 may additionally determine whether a level of the receiving signal is greater than or equal to a reference level using an output of the differential amplifier 400. In FIG. 4, the biosignal receiving device 20 according to another embodiment of the present disclosure may additionally provide a criterion for selecting an optimal combination of receiving electrodes based on a reference voltage level in addition to the correlation value, thereby further improving the communication performance of the human body communication system.

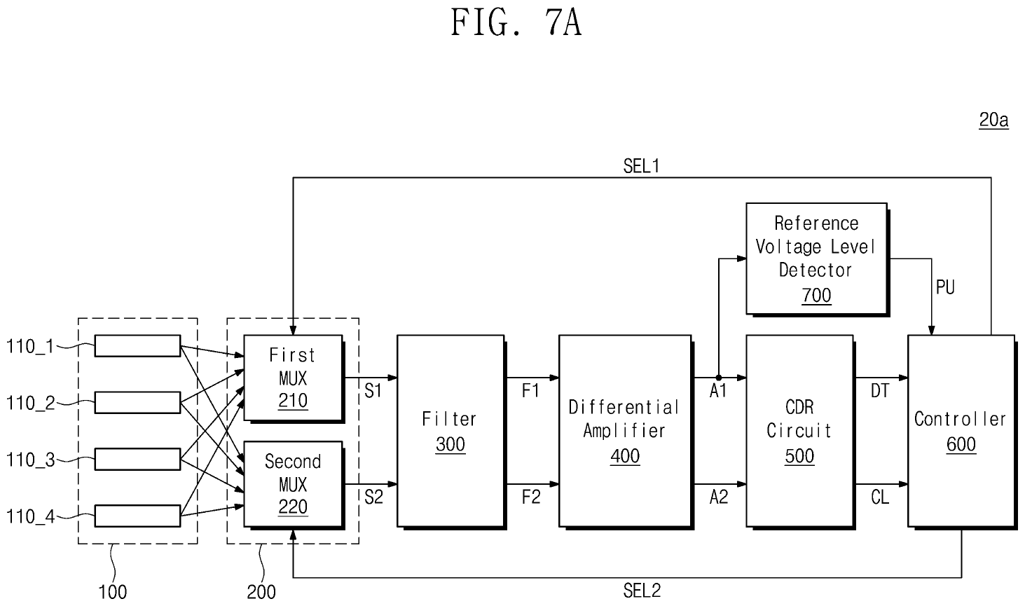

The reference voltage level detector 700 may receive a first amplified signal Al from the differential amplifier 400, and may generate a pulse signal PU based thereon. The reference voltage level detector 700 may provide the generated pulse signal PU to the controller 600 (refer to FIG. 1), and the detailed configuration and operation of the reference voltage level detector 700 will be described with reference to FIG. 6 to be described later.

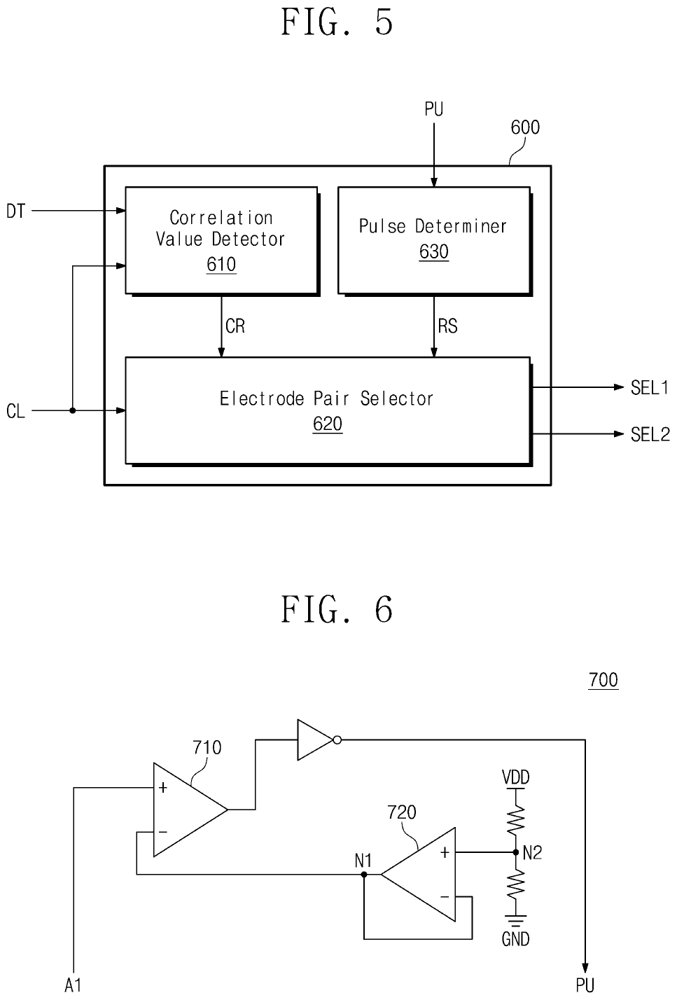

FIG. 5 is a block diagram illustrating the controller 600 included in the biosignal receiving device 20 (refer to FIG. 4) illustrated in FIG. 4. Hereinafter, descriptions of components and operations of the biosignal receiving device 10 according to the embodiment of the present disclosure described above with reference to FIGS. 1 to 4 will be omitted to avoid redundancy. Referring to FIG. 5, unlike the controller 600 included in the biosignal receiving device 10 illustrated in FIG. 1, the controller 600 included in the biosignal receiving device 20 illustrated in FIG. 4 may further include a pulse determiner 630.

The pulse determiner 630 may determine a goodness degree of a level of the receiving signal based on the pulse signal PU provided from the reference voltage level detector 700 (refer to FIG. 4). The goodness degree of the level of the receiving signal may be determined depending on the level of the pulse signal PU. For example, when the pulse signal PU has a high level (1), it may mean that the level of the receiving signal is good and the goodness degree is high, and when the pulse signal PU has a low level (0), it may mean that the level of the receiving signal is not good and the goodness degree is low.

The pulse determiner 630 may provide a determination result signal RS including information on the determination result with respect to the goodness degree of the level of the receiving signal to the electrode pair selector 620. Accordingly, the electrode pair selector 620 may select a combination of receiving electrodes that may exhibit the optimal communication performance in the biosignal receiving device 20, based on the correlation value derived from the correlation value detector 610 and the result of whether the signal level provided from the pulse determiner 630 is good.

FIG. 6 is a block diagram illustrating the reference voltage level detector 700 included in the biosignal receiving device 20 (refer to FIG. 4) illustrated in FIG. 4. The reference voltage level detector 700 may include a comparator 710 and an amplifier 720.

The comparator 710 may compare the receiving signal with the reference voltage to compare an amplitude of the receiving signal with a level of the reference voltage. A first amplified signal Al provided from the differential amplifier 400 (refer to FIG. 4) may be input to a positive input (+) of the comparator 710, and the reference voltage provided from the amplifier 720 may be input to a negative input (−) of the comparator 710. The level of the reference voltage may be the same as a voltage applied to a first node N1.

The amplifier 720 may amplify a voltage applied to a second node N2 positioned between a supply voltage VDD and the ground GND. The reference voltage level detector 700 may generate the pulse signal PU based on the comparison result derived from the comparator 710, and the generated pulse signal PU may be provided to the controller 600 (refer to FIG. 4).

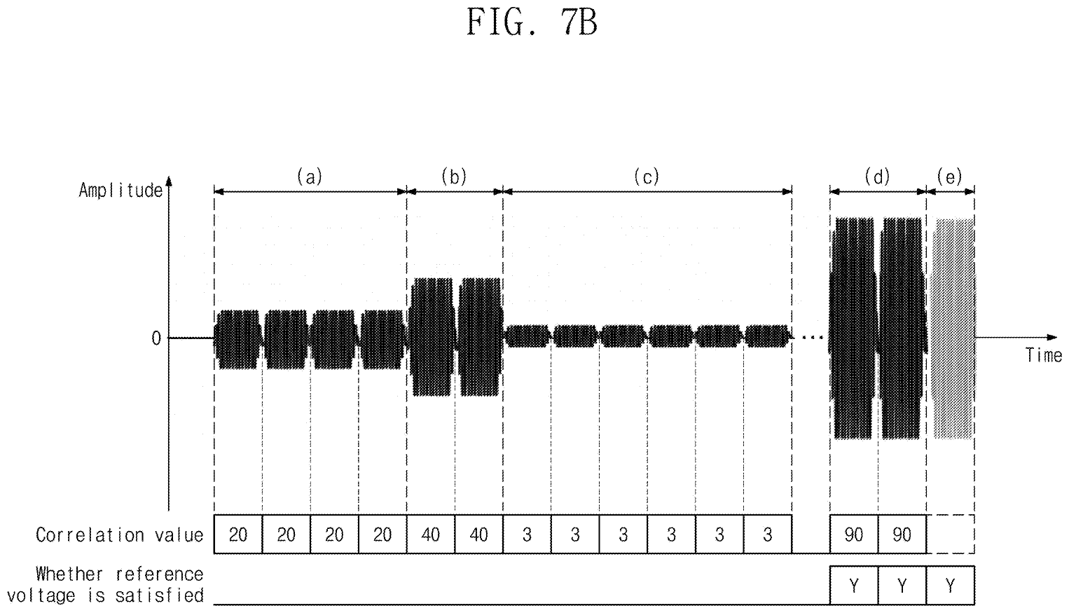

FIG. 7A is a diagram illustrating an embodiment 20a in which the four receiving electrodes 110_1, 110_2, 110_3, and 110_4 are included in the biosignal receiving device 20 (refer to FIG. 4) illustrated in FIG. 4. FIG. 7B is a diagram for describing a principle of selecting a receiving electrode combination in the biosignal receiving device 20 illustrated in FIG. 7A. Hereinafter, descriptions of components and operations of the biosignal receiving device 10 or 20 according to the embodiment of the present disclosure described above with reference to FIGS. 1 to 6 will be omitted to avoid redundancy.

There are 12 receiving electrode pairs (the first electrode and the second electrode) that may occur in the embodiment 20a in which the four receiving electrodes 110_1, 110_2, 110_3, and 110_4 illustrated in FIG. 7A are included. Accordingly, the biosignal receiving device 10 according to an embodiment of the present disclosure may select a receiving electrode pair capable of exhibiting optimal communication performance among 12 cases.

FIG. 7B illustrates a waveform received by the biosignal receiving device 20 according to an embodiment of the present disclosure. In more detail, FIG. 7B illustrates a waveform of the receiving signal received from some of the receiving electrode pairs among the receiving electrode pairs (the first electrode and the second electrode) that may occur in the embodiment 20a in which the four receiving electrodes 110_1, 110_2, 110_3, and 110_4 illustrated in FIG. 7A are included. In a graph illustrating the waveform of the receiving signal, an x-axis means a time, and a y-axis means an amplitude of the receiving signal.

For example, in FIG. 7B, the signal in section (a) may illustrate a waveform of the signal received from the first receiving electrode 110_1 and the second receiving electrode 110_2, and the signal in section (b) may illustrate a waveform of the signal received from the first receiving electrode 110_1 and the fourth receiving electrode 110_4. The signal in section (c) may illustrate a waveform of the signal received from the second receiving electrode 110_2 and the first receiving electrode 110_1, and the signal in section (d) may illustrate a waveform of the signal received from the third receiving electrode 110_3 and the fourth receiving electrode 110_4. The signal in section (e) may be a data signal.

In the embodiment 20a in which the four receiving electrodes 110_1, 110_2, 110_3, and 110_4 illustrated in FIG. 7A are included, the process of selecting the optimal receiving electrode combination based on the correlation value is the same as that disclosed in FIGS. 3A and 3B. However, the embodiment 20a including the four receiving electrodes 110_1, 110_2, 110_3, and 110_4 illustrated in FIG. 7A may further improve the performance of the communication system, by additionally determining whether the level of the signal is equal to or greater than the level the reference voltage in selecting the optimal receiving electrode combination, and by selecting the optimal receiving electrode combination based on the determination.

As illustrated in FIG. 3B, it is determined that the optimal receiving electrode combination based on the correlation value is in section (d). In addition, since the level of the signal in section (d) is determined to be equal to or greater than the level of the reference voltage, in this case, the combination of the third receiving electrode 110_3 and the fourth receiving electrode 110_4 may be selected as an optimal receiving electrode pair. Determination of whether the correlation value and the reference voltage are satisfied may be performed simultaneously in parallel or may be performed sequentially. In addition, when the correlation value has a magnitude greater than or equal to the threshold correlation value, it is possible to determine whether the reference voltage is satisfied only for the receiving electrode combination having a magnitude greater than or equal to the threshold correlation value, or whether the reference voltage is satisfied for all receiving electrode pairs.

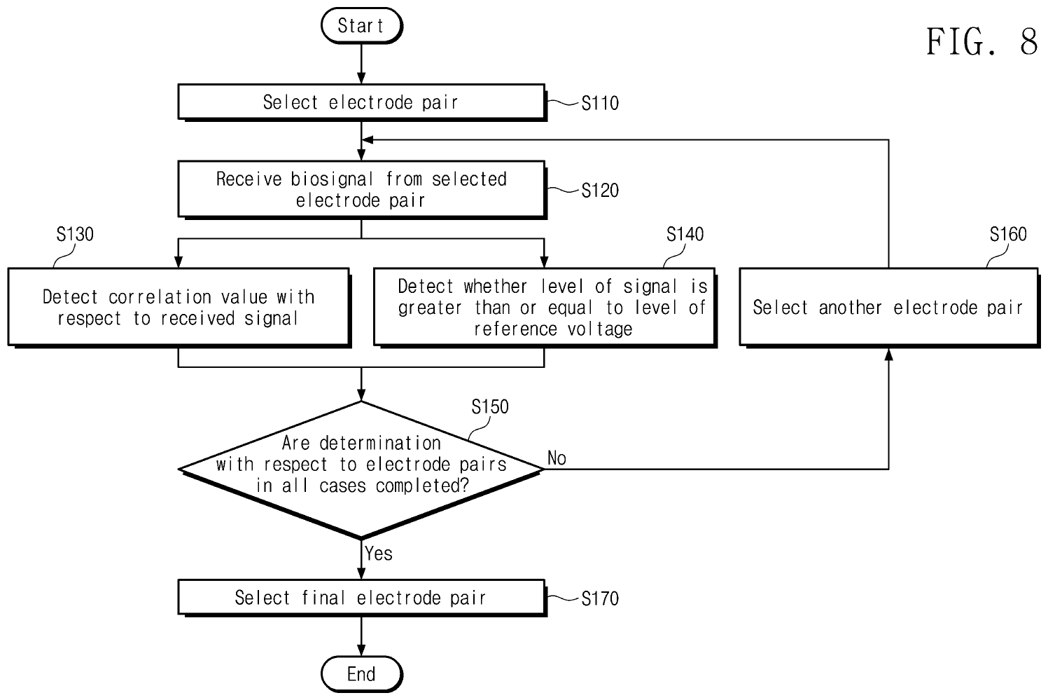

FIG. 8 is a diagram illustrating a method of receiving a biosignal according to an embodiment of the present disclosure. Hereinafter, descriptions of components and operations of the biosignal receiving device 10 or 20 according to the embodiment of the present disclosure described above with reference to FIGS. 1 to 7B will be omitted to avoid redundancy.

In operation S110, the biosignal receiving device 10 or 20 (refer to FIGS. 1 and 4) according to the present disclosure may select an arbitrary electrode pair from among the plurality of receiving electrodes 110_1, 110_2, . . . , and 110_n.

In operation S120, the biosignal receiving device 10 or 20 may receive a biosignal from an arbitrarily selected electrode pair. The biosignal refers to a signal transmitted from a receiving device included in a human body communication system to an electrode pair using the human body as a medium. The noise in the biosignal may be removed through the filter 300 (refer to FIG. 1), the amplitude of the biosignal may be amplified through the differential amplifier 400 (refer to FIG. 1), and the data signal DT (refer to FIG. 1) and the clock signal CL (refer to FIG. 1) may be detected through the CDR circuit 500 (refer to FIG. 1) based on this.

In operation S130, the biosignal receiving device 10 or 20 may detect a correlation value with respect to a signal received from an arbitrarily selected electrode pair based on the data signal DT and the clock signal CL. The correlation value may correspond to a match rate between the preamble of the transmitting signal transmitted from the transmitting device and the preamble of the receiving signal received by the biosignal receiving device 10 or 20.

In operation S140, the biosignal receiving device 10 or 20 may detect whether a level of a signal received from an arbitrarily selected electrode pair is greater than or equal to a level of a reference voltage. In FIG. 8, operations S130 and S140 are illustrated as being performed in parallel, but this is only one embodiment and may be performed sequentially.

In operation S150, the biosignal receiving device 10 or 20 may determine whether a determination on the correlation value is performed and a determination as to whether it is greater than or equal to the reference voltage is performed with respect to electrode pairs that may be generated from all the receiving electrodes included in the biosignal receiving device 10 or 20. When determination is not made with respect to all possible receiving electrode pairs, a procedure may proceed to operation S160. When determination is made with respect to all possible receiving electrode pairs, a procedure may proceed to operation S170.

In operation S160, the biosignal receiving device 10 or 20 may select one of the remaining combinations except for the previously selected electrode pair.

When another electrode pair is selected, the procedure may return to operation S120.

In operation S170, the biosignal receiving device 10 or 20 may select a final electrode pair capable of optimizing communication performance, and the procedure ends.

In the present specification, in a communication system using the human body as a medium, the biosignal receiving device 10 or 20 is disclosed that the plurality of receiving electrodes 110_1, 110_2, . . . , and 110_n operate as a path of the receiving signal in the receiving electrode unit 100 (refer to FIG. 1). However, the spirit of the present disclosure is not limited thereto, and may be extended and applied to a wireless communication system including a plurality of receiving signal paths. For example, the spirit of the present disclosure may be applied to a wireless communication system including a plurality of antennas as a plurality of receiving signal paths.

According to an embodiment of the present disclosure, the device and method for receiving the biosignal may overcome performance degradation due to attenuation of a waveform of a receiving signal and may improve signal receiving performance.

The above description refers to embodiments for implementing the present disclosure. Embodiments in which a design is changed simply or which are easily changed may be included in the present disclosure as well as an embodiment described above. In addition, technologies that are easily changed and implemented by using the above embodiments may be included in the present disclosure. While the present disclosure has been described with reference to embodiments thereof, it will be apparent to those of ordinary skill in the art that various changes and modifications may be made thereto without departing from the spirit and scope of the present disclosure as set forth in the following claims.

What is claimed is:

1. A biosignal receiving device used in a communication system using a human body as a medium, comprising:
a receiving electrode unit including a plurality of receiving electrodes for receiving a biosignal received from the human body;
an input selection unit including a first MUX for selecting a first electrode from among the plurality of receiving electrodes and a second MUX for selecting a second electrode different from the first electrode from among the plurality of receiving electrodes, and configured to output a first biosignal received by the first electrode and a second biosignal received by the second electrode;
a filter configured to remove noise included in the first biosignal and the second biosignal, and output a first filtered signal and a second filtered signal from which the noise is removed;
a differential amplifier configured to amplify a difference between the first filtered signal and the second filtered signal and to output a first amplified signal and a second amplified signal;
a CDR circuit configured to generate a data signal and a clock signal from the first amplified signal and the second amplified signal, and to output the data signal and the clock signal; and
a controller configured to output a first selection signal for selecting the first electrode from among the plurality of receiving electrodes and a second selection signal for selecting the second electrode from among the plurality of receiving electrodes to the input selection unit, based on the data signal and the clock signal.

2. The biosignal receiving device of claim 1, wherein the controller includes:
a correlation value detector configured to detect a correlation value of the biosignal based on the data signal and the clock signal and to output the correlation value; and
an electrode pair selector configured to generate the first selection signal and the second selection signal based on the clock signal and the correlation value.

3. The biosignal receiving device of claim 2, wherein the correlation value detector compares a preamble of a transmitting signal transmitted from a transmitting device included in a communication system using the human body as the medium with a preamble of the biosignal to derive the correlation value.

4. The biosignal receiving device of claim 3, wherein, when specific electrodes among the plurality of electrodes are selected as the first electrode and the second electrode, and the correlation value exceeds a predetermined threshold correlation value, the specific electrodes are finally selected as the first electrode and the second electrode.

5. The biosignal receiving device of claim 1, further comprising:
a reference voltage level detector configured to determine whether a level of the biosignal is equal to or greater than a level of a reference voltage based on the first amplified signal, and to output a pulse signal to the controller based on the determination result.

6. The biosignal receiving device of claim 5, wherein the controller generates, based on the pulse signal, a result signal indicating a result of determining whether the biosignal has the level greater than or equal to the reference voltage, and outputs the result signal to the electrode pair selector.

7. The biosignal receiving device of claim 6, wherein the electrode pair selector generates the first selection signal and the second selection signal based on the correlation value and the result of the determining.

8. The biosignal receiving device of claim 5, wherein a reference voltage level detector includes:
an amplifier configured to amplify and output a voltage applied to a node between a supply voltage and a ground; and
a comparator configured to compare the first amplified signal with a reference voltage output from the amplifier and to output the pulse signal corresponding to the comparison result.

9. The biosignal receiving device of claim 8, wherein the pulse signal has a high level when the level of the first amplified signal is greater than or equal to the reference voltage, and has a low level when the level of the first amplified signal is less than the reference voltage.

10. A biosignal receiving method comprising:
selecting a first electrode and a second electrode from among a plurality of receiving electrodes;
receiving a biosignal from the first electrode and the second electrode;
detecting a correlation value with respect to the biosignal; and
determining whether a level of the biosignal is greater than or equal to a level of a reference voltage, and
wherein the correlation value is based on a correlation between a preamble of a transmitting signal transmitted to a human body providing the biosignal and a preamble of the biosignal.

11. The biosignal receiving method of claim 10, further comprising:
determining whether the correlation value is detected for all of receiving electrode combinations generated from the plurality of receiving electrodes.

12. The biosignal receiving device of claim 11, further comprising:
when the correlation value is not detected for all of the receiving electrode combinations, reselecting the combination of the first electrode and the second electrode from receiving electrode combinations that are not selected among the receiving electrode combinations.

13. The biosignal receiving device of claim 10, further comprising:
determining whether the correlation value is greater than or equal to a predetermined threshold correlation value.
